(19) United States
(12) Patent Application Publication
(10) Pub. No.: US 2016/0132632 A1
(43) Pub. Date: May 12, 2016

(54) System, Method and Software for Improved Drug Efficacy and Safety in a Patient

(71) Applicant: Insilico Medicine, Inc., Baltimore, MD (US)

(72) Inventors: Aleksandrs Zavoronkovs, London (GB); Anton Buzdin, Istra (RU); Nikolay Borisov, Krasnoznamensk (RU); Alexander Aliper, Ramenskoye (RU)

(21) Appl. No.: 14/935,461

(22) Filed: Nov. 9, 2015

Related U.S. Application Data
(60) Provisional application No. 62/077,330, filed on Nov. 10, 2014.

(51) Int. Cl. G06F 19/12; C12Q 1/68

(52) U.S. Cl. CPC G06F 19/12; C12Q 1/6886; C12Q 2600/158; C12Q 2600/106

(57) ABSTRACT

The present invention provides systems, methods and software predicting drug efficacy for treating a disorder in a patient, the method including providing a drug score database (DSD) based on pathway manifestation strengths (PMSs) for a plurality of biological pathways associated with the drug in the treatment of the disorder and comparing the pathway manifestation strengths of the plurality of biological pathways of the patient with the drug score database to provide a predictive indication if the patient is a responder or non-responder to the drug to determine whether the drug should be used in treating the patient.

### FIELD OF THE INVENTION

The present invention relates generally to systems and methods of analysis of gene signaling pathways, and more specifically to systems and methods for improving efficacy and safety of drug combinations in a patient.

### BACKGROUND OF THE INVENTION

In the twentieth century, enormous strides were made in combatting infectious diseases, in their detection and drugs to treat them. The major problem in the medical world has thus shifted from treating acute diseases to treating chronic diseases. Over the last few decades, with the advent of genetic engineering, much research and funding has been invested in genomics and gene-based personalized medicine. A need has arisen to develop diagnostic tools for use in the characterization of personalized aspects of chronic diseases and diseases associated with aging.

Novel methods have been developed for screening for drugs that can minimize the difference between the various cellular or tissue states in a variety of tissues, while also taking into accounting for toxicity and adverse effect of the drug.

Intracellular signaling pathways (SPs) regulate numerous processes involved in normal and pathological conditions including development, growth, aging and cancer. Many bioinformatic tools have been developed, which analyze SPs.

The information relating to signaling pathway activation (SPA) can be obtained from the massive proteomic or transcriptomic data. Although the proteomic level may be somewhat closer to the biological function of SPA, the transcriptomic level of studies today is far more feasible in terms of performing experimental tests and analyzing the data.

US2008254497A provides a method of determining whether tumor cells or tissue is responsive to treatment with an ErbB pathway-specific drug. In accordance with the invention, measurements are made on such cells or tissues to determine values for total ErbB receptors of one or more types, ErbB receptor dimers of one or more types and their phosphorylation states, and/or one or more ErbB signaling pathway effector proteins and their phosphorylation states. These quantities, or a response index based on them, are positively or negatively correlated with cell or tissue responsiveness to treatment with an ErbB pathway-specific drug. In one aspect, such correlations are determined from a model of the mechanism of action of a ErbB pathway-specific drug on an ErbB pathway. Preferably, methods of the invention are implemented by using sets of binding compounds having releasable molecular tags that are specific for multiple components of one or more complexes formed in ErbB pathway activation. After binding, molecular tags are released and separated from the assay mixture for analysis.

U.S. Pat. No. 8,623,592 discloses methods for treating patients which methods comprise methods for predicting responses of cells, such as tumor cells, to treatment with therapeutic agents. These methods involve measuring, in a sample of the cells, levels of one or more components of a cellular network and then computing a Network Activation State (NAS) or a Network Inhibition State (NIS) for the cells using a computational model of the cellular network. The response of the cells to treatment is then predicted based on the NAS or NIS value that has been computed. The invention also comprises predictive methods for cellular responsiveness in which computation of a NAS or NIS value for the cells (e.g., tumor cells) is combined with use of a statistical classification algorithm. Biomarkers for predicting responsiveness to treatment with a therapeutic agent that targets a component within the ErbB signaling pathway are also provided.

There thus remains a need for systems and methods, which can predict drug efficacy of drug combinations in a patient. There further remains a need for systems and methods, which can predict drug combination adverse effects. There also remains a need for systems and methods, which can predict and maximize drug combination positive pathway activation.

### SUMMARY OF THE INVENTION

It is an object of some aspects of the present invention to provide systems and methods, for improving efficacy and safety of drug combinations in a patient.

It is a further object of some aspects of the present invention to provide systems and methods, which provide an indication if a drug combination is likely to be effective in a geriatric or aging patient.

It is a further object of some aspects of the present invention to provide systems and methods, which provide an indication if a drug combination is likely to induce adverse effects in a geriatric or aging patient.

It is another further object of some aspects of the present invention to provide systems and methods, which provide an indication if a drug combination is likely to induce positive pathway activation in a geriatric or aging patient.

The present invention provides methods for screening for new drug candidates and for re-purposing the approved drugs and combinations by estimating their ability to suppress pathologically activated or down-regulated biological pathways.

The present invention also provides methods for screening for drugs that can minimize the difference between the various cellular or tissue states in a variety of tissues, while also taking into accounting for toxicity and adverse effect of the drug.

The present invention further provides methods for screening for combinations of drugs that can minimize the difference between the various cellular or tissue states in a variety of tissues, while also taking into accounting for toxicity and adverse effect of the drug.

The present invention further provides methods for screening for new drug candidates and for re-purposing the approved drugs and combinations by estimating their ability to suppress pathologically activated or down-regulated biological pathways, while activating beneficial pathways in the patient.

The present invention further provides methods for providing an individual with a drug or drug combination, which is personalized to minimize adverse effects and maximize beneficial pathway activation in that specific patient.

In general, the methods of the present invention are operative to:
a) Obtain many tissues from a patient and generate tissue-specific gene expression data.
b) Optionally obtain nucleic acids data from at least one blood sample from the patient.
c) Compare gene expression data from the patient with at least one of:
   i. healthy patients gene expression data;
   ii. young patients gene expression data; and
   iii. results of the previous tissue-specific gene expression analysis from the same patient.
d) Compare pathway activation profiles (PAS) on a tissue specific level using methods described in Buzdin et al., 2014.
e) Analyze which pathways are pathological and which pathways are good/beneficial for the cell/the patient.
f) For the pathways that are pathological, an optimal combination of drugs is determined. This may be performed by using drugs with known or predicted molecular targets or effects on transcriptomes, which minimize the difference between the different pathway activation states.
g) The optimal combination of drugs from the previous step is further analyzed to minimize toxicity and adverse effects, such that combinations which work best in that specific patient and have minimal toxicity and adverse effects in most tissues (especially in the long-lived cells) are chosen for the specific patient. When screening for geroprotector (drugs that slow or prevent the pathologic age-related changes or repair accumulated damage) the combination must work well in long-lived cells and tissues like the brain, muscle, stem cells.

There is thus provided according to an embodiment of the present invention, a method for improving drug efficacy and safety for treating a disorder in a patient, the method including;
a. providing a drug score database (DSD) based on pathway manifestation strengths (PMSs) for a plurality of biological pathways associated with the drug in the treatment of the disorder; and
b. comparing the pathway manifestation strengths of the plurality of biological pathways of the patient with the drug score database to provide a predictive indication if the patient is a responder or non-responder to the drug to determine whether the drug should be used in treating the patient.

Furthermore, according to an embodiment of the present invention, the providing a drug score database (DSD) step includes;
c. obtaining proliferative bodily samples and healthy bodily samples from patients;
d. applying the drug to the patients; and
e. determining responder and non-responder patients to the drug.

Additionally, according to an embodiment of the present invention, the determining step includes comparing gene expression in selected signaling pathways.

Moreover, according to an embodiment of the present invention, the selected signaling pathways are associated with the drug.

Further, according to an embodiment of the present invention, the determining step further includes determining a drug score at least one pathway manifestation strength (PMS) value for each pathway in the responder and the non-responder patients.

Yet further, according to an embodiment of the present invention, the determining step further includes determining a drug score for the drug based on the at least one pathway manifestation strength (PMS) value.

Furthermore, according to an embodiment of the present invention, the bodily samples are selected from the group consisting of a tissue sample, a cell culture, an individual single cell, a bodily sample, an organism sample and a microorganism sample.

Notably, according to an embodiment of the present invention the biological pathways are signaling pathways.

Furthermore, according to an embodiment of the present invention, the biological pathways are metabolic pathways.

Additionally, according to an embodiment of the present invention, the gene expression includes quantifying expression of plurality of gene products.

Further, according to an embodiment of the present invention, the gene products include a set of at least five gene products.

Furthermore, according to an embodiment of the present invention, the method further includes;
d. calculating a pathway activation strength (PAS), indicative of the pathway activation of each of the biological pathways.

Furthermore, according to an embodiment of the present invention, the calculating step includes adding concentrations of the set of the at least five gene products of the sample and comparing to a same set in the at least one control sample.

Moreover, according to an embodiment of the present invention, the gene products provide at least one function in the biological pathway.

Additionally, according to an embodiment of the present invention, the at least one function includes an activation function and a suppressor function.

Further, according to an embodiment of the present invention, the at least one function includes an up-regulating function and a down-regulating function.

Yet further, according to an embodiment of the present invention, the determining step includes at least one of profiling gene expression, RNA profiling, RNA sequencing, DNA profiling, DNA sequencing, protein profiling, amino acid sequencing, at least one immunochemical methodology, a mass spectrometry analysis, a microarray technology, a quantitative PCR methodology and combinations thereof.

Furthermore, according to an embodiment of the present invention, the method is quantitative. Additionally or alternatively, the method is qualitative.

Further, according to an embodiment of the present invention, A the patients are sick.

Furthermore, according to an embodiment of the present invention, the sick subject suffers from a proliferative disease or disorder.

Importantly, according to an embodiment of the present invention, the proliferative disease or disorder is cancer.

Furthermore, according to an embodiment of the present invention, the proliferative disease or disorder is colon cancer.

Additionally, according to an embodiment of the present invention, the drug is a monoclonal antibody (mAb).

Furthermore, according to an embodiment of the present invention, the monoclonal antibody (mAb) is Bevacizumab.

Moreover, according to an embodiment of the present invention, the pathway is selected from the group consisting of a Caspase Cascade pathway; a CREB pathway; a GPCR pathway; a CSK3 pathway; an HIF1Alpha pathway; an HIF1Alpha Pathway VEGF pathway; an ILK pathway; an IP3 pathway; a mAb pathway; a PPAR pathway; a VEGF pathway; and combinations thereof.

There is thus provided according to an embodiment of the present invention, a computer software product, the product configured for predicting drug efficacy for treating a disorder in a patient, the product including a computer-readable medium in which program instructions are stored, which instructions, when read by a computer, cause the computer to;
a. provide a drug score database (DSD) based on pathway manifestation strengths (PMSs) for a plurality of biological pathways associated with the drug in the treatment of the disorder; and
b. compare the pathway manifestation strengths of the plurality of biological pathways of the patient with the drug score database to provide a predictive indication if the patient is a responder or non-responder to the drug to determine whether the drug should be used in treating the patient.

There is thus provided according to another embodiment of the present invention, a system for predicting drug efficacy for treating a disorder in a patient the system including;
a. a processor adapted to activate a computer-readable medium in which program instructions are stored, which instructions, when read by a computer, cause the processor to;
   i. provide a drug score database (DSD) based on pathway manifestation strengths (PMSs) for a plurality of biological pathways associated with the drug in the treatment of the disorder; and
   ii. compare the pathway manifestation strengths of the plurality of biological pathways of the patient with the drug score database to provide a predictive indication if the patient is a responder or non-responder to the drug to determine whether the drug should be used in treating the patient;
b. a memory for storing the drug score database (DSD); and
c. a display for displaying data associated with the predictive indication of the patient.

Furthermore, according to an embodiment of the present invention, the drug, previously used for a first indication is used for a new second indication.

Importantly, according to an embodiment of the present invention, the drug is at least one of repurposed and repositioned.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a simplified schematic illustration of a system for improving efficacy and safety of drug or drug combinations in a patient, in accordance with an embodiment of the present invention;

FIG. 1B is a schematic showing further details of drug profile database and transcriptomic database of FIG. 1A, in accordance with an embodiment of the present invention;

FIGS. 2A-2D are simplified schematic steps in a method for improving efficacy and safety of a drug or drug combination in a patient, in accordance with an embodiment of the present invention; and

FIGS. 3A-3B are simplified diagrams of effects of a drug on up-regulating and down-regulating signaling and metabolic pathways, respectively, in accordance with embodiments of the present invention.

In all the figures similar reference numerals identify similar parts.

### DETAILED DESCRIPTION OF PREFERRED EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

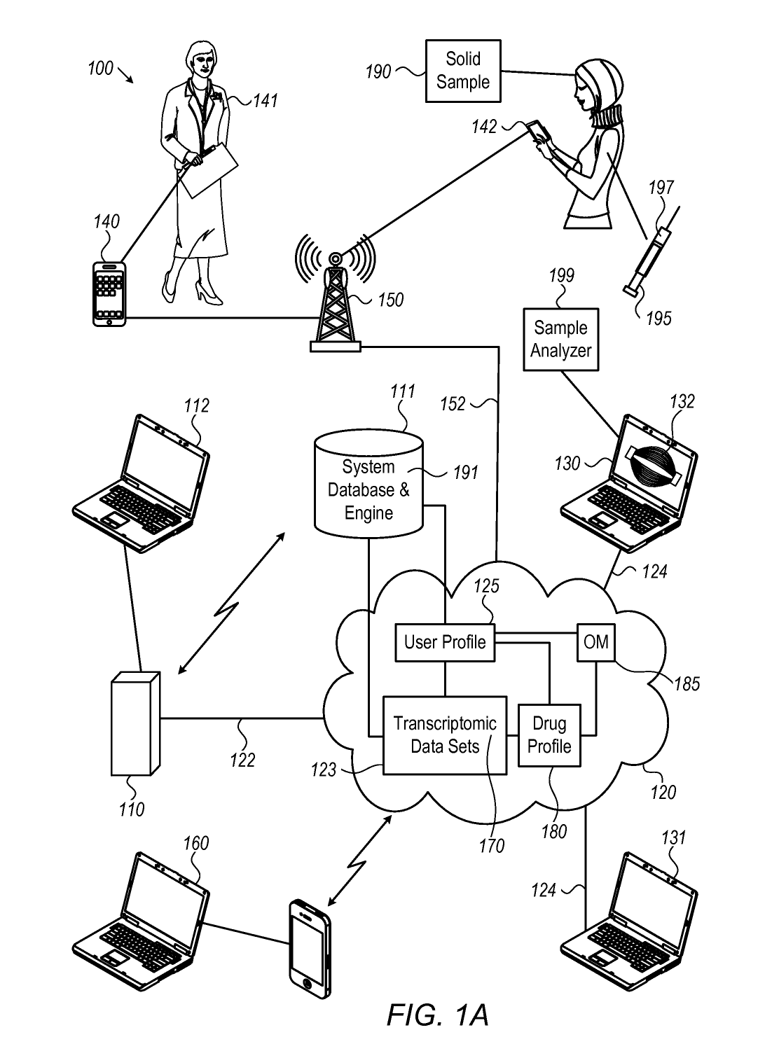

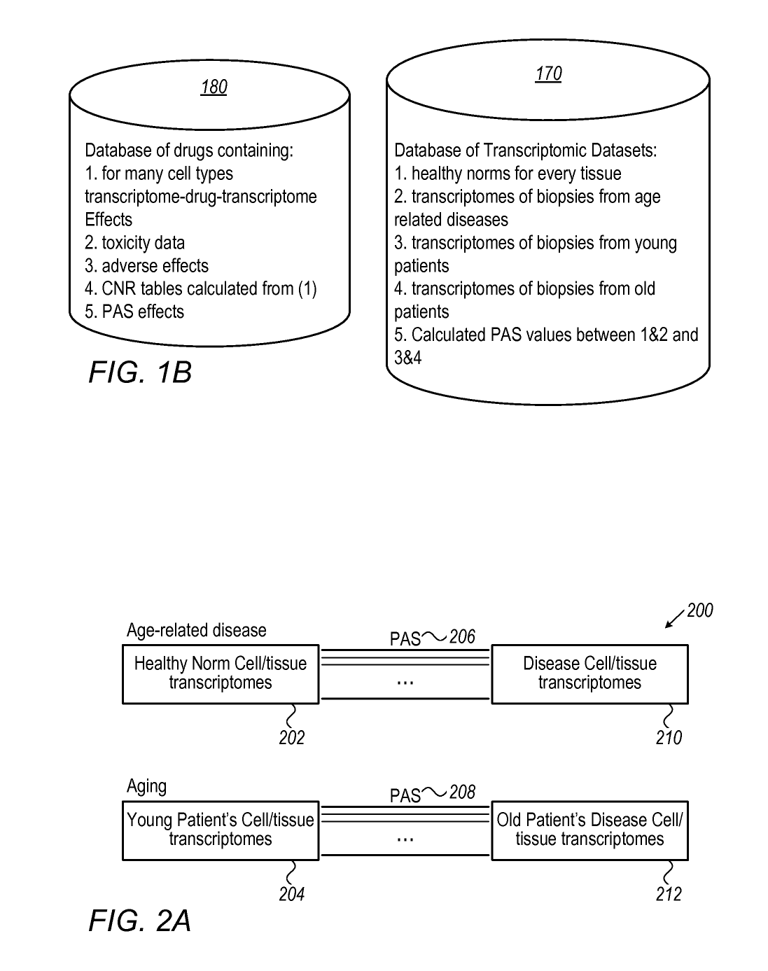

Reference is now made to FIG. 1A, which is a simplified schematic illustration of a system for improving efficacy and safety of drug combinations in a patient, in accordance with an embodiment of the present invention.

System 100 typically includes a server utility 110, which may include one or a plurality of servers and one or more control computer terminals 112 for programming, trouble-shooting servicing and other functions. Server utility 110 includes a system engine 111 and database, 191. Database 191 comprises a user profile database 125, a pathway cloud database 123 and a drug profile database 180.

Depending on the capabilities of a mobile device, system 100 may also be incorporated on a mobile device that synchronizes data with a cloud-based platform.

The drug profile database comprises data relating to a large number of drugs for controlling and treating ageing processes. For each type of drug, the dosage values, pharmo-kinetic data and profile, pharmodynamic data and profiles are included.

The drug profile database further comprises data of drug combinations, including dosage values pharmo-kinetic data and profile, pharmodynamic data and profiles.

A medical professional, research personnel or patient assistant/helper/carer 141 is connected via his/her mobile device 140 to server utility 110. The patient, subject or child 143 is also connected via his/her mobile device 142 to server utility 110. In some cases, the subject may be a mammalian subject, such as a mouse, rat, hamster, monkey, cat or dog, used in research and development. In other cases, the subject may be a vertebrate subject, such as a frog, fish or lizard. The patient or child is monitored using a sample analyzer 199. Sample analyzer 199, may be associated with one or more computers 130 and with server utility 110. Computer 130 and/or sample analyzer 199 may have software therein for predicting drug efficacy in a patient, as will be described in further details hereinbelow.

Typically, gene expression data 123 (FIG. 1), generated by the software of the present invention, is stored locally and/or in cloud 120 and/or on server 110.

The sample analyzer may be constructed and configured to receive a solid sample 190, such as a biopsy, a hair sample or other solid sample from patient 143, and/or a liquid sample 195, such as, but not limited to, urine, blood or saliva sample. The sample may be extracted by any suitable means, such as by a syringe 197.

The patient, subject or child 143 may be provided with a drug (not shown) by health professional/research/doctor 141.

System 100 further comprises an outputting module 185 for outputting data from the database via tweets, emails, voicemails and computer-generated spoken messages to the user, carers or doctors, via the Internet 120 (constituting a computer network), SMS, Instant Messaging, Fax through link 122.

Users, patients, health care professionals or customers 141, 143 may communicate with server 110 through a plurality of user computers 130, 131, or user devices 140, 142, which may be mainframe computers with terminals that permit individual to access a network, personal computers, portable computers, small hand-held computers and other, that are linked to the Internet 120 through a plurality of links 124. The Internet link of each of computers 130, 131, may be direct through a landline or a wireless line, or may be indirect, for example through an intranet that is linked through an appropriate server to the Internet. System 100 may also operate through communication protocols between computers over the Internet which technique is known to a person versed in the art and will not be elaborated herein.

Users may also communicate with the system through portable communication devices such as mobile phones 140, communicating with the Internet through a corresponding communication system (e.g. cellular system) 150 connectable to the Internet through link 152. As will readily be appreciated, this is a very simplified description, although the details should be clear to the artisan. Also, it should be noted that the invention is not limited to the user-associated communication devices—computers and portable and mobile communication devices—and a variety of others such as an interactive television system may also be used.

The system 100 also typically includes at least one call and/or user support and/or tele-health center 160. The service center typically provides both on-line and off-line services to users. The server system 110 is configured according to the invention to carry out the methods of the present invention described herein.

It should be understood that many variations to system 100 are envisaged, and this embodiment should not be construed as limiting. For example, a facsimile system or a phone device (wired telephone or mobile phone) may be designed to be connectable to a computer network (e.g. the Internet). Interactive televisions may be used for inputting and receiving data from the Internet. Future devices for communications via new communication networks are also deemed to be part of system 100. Memories may be on a physical server and/or in a virtual cloud.

A mobile computing device may also embody a non-synced or offline copy of memories, copies of pathway cloud data, user profiles database, drug profiles database and execute the system, engine locally.

FIG. 1B is a schematic 120 showing further details of drug profile database 180 and transcriptomic database 170 of FIG. 1A, in accordance with an embodiment of the present invention.

Drug profile database 180 comprises:

1. For many cell types, transcriptome-drug-transcriptome effects;

2. Drug toxicity data;

3. Drug adverse effects

4. Drug CNR tables calculated as described in reference (1);

5. Drug pathway activation strength (PAS) effects.

Database of Transcriptomic Datasets 170 comprises:

1. Healthy norms for every tissue;

2. Transcriptomes of biopsies from age-related diseases;

3. Transcriptomes of biopsies from young patients;

4. Transcriptomes of biopsies from old patients; and

5. Calculated PAS values from 1 & 2 above and 3 & 4 above.

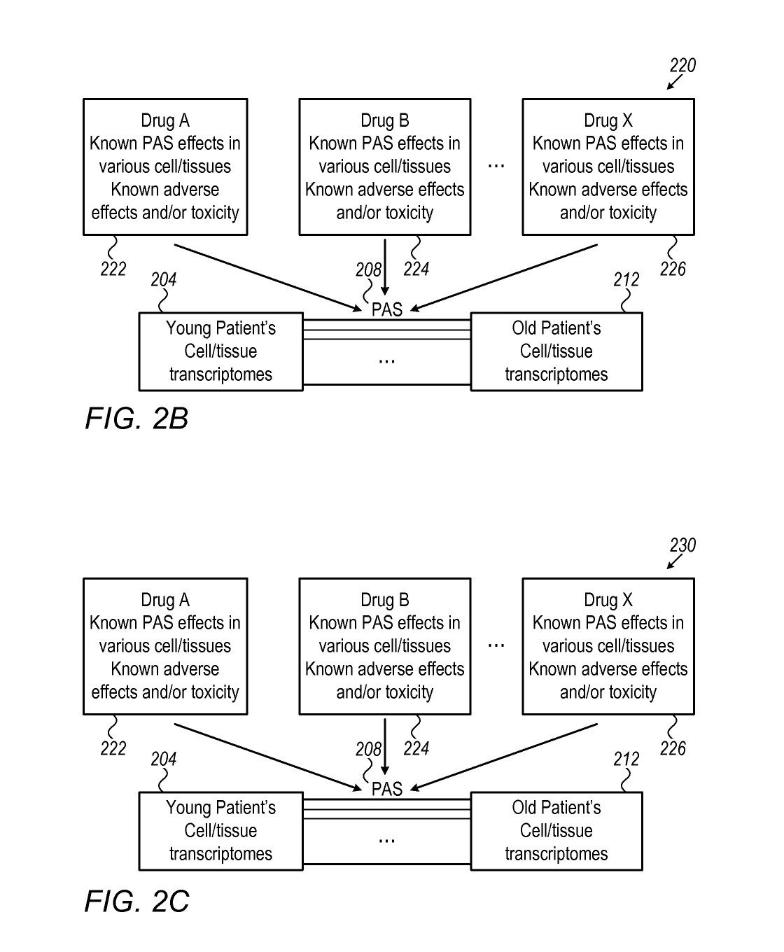

Reference is now made to FIGS. 2A-2D are respective simplified schematic steps 200, 220, 230 and 240 in a method for improving efficacy and safety of a drug or drug combination in a patient, in accordance with an embodiment of the present invention.

Step 1 (FIG. 2A): For every available tissue type construct a set of pathways considered to be pathologic. Compare healthy/normal cell tissue transcriptomes 202 with disease cell/tissue transcriptomes 210 to generate pathway activation strengths 206 for a first set of pathways.

Step 2 (FIG. 2B): Calculate the net effect of the combination on PAS by utilizing known PAS effects

For each drug, such as drug A 222, drug B 224, up to drug X, 226 calculate the PAS effects in cell/tissues known for adverse effects and/or toxicity, the combination of drugs must minimize the signaling disturbance by minimizing PAS 208 of sets of pathological pathways without significant effects of beneficial and protective pathways. Compare young patient's cell/tissue transcriptomes 204 with old patient's disease cell/tissue transcriptomes 212 to generate pathway activation strengths 208 for a second set of pathways.

Step 3 (FIG. 2C): Evaluate the net toxicity and adverse effects for each prospective combination.

Step 4 (FIG. 2D): Rank the combinations in a ranking step 242 by the ability to minimize PAS differences, adverse effects and toxicity.

The steps in the method of the present invention may optionally include any one or more of the following: For many tissues from a patient, tissue-specific gene expression data are determined N nucleic acids are extracted from blood.
1. Gene expression data with (three cases): a. healthy patients b. young patients c. results of the previous tissue-specific gene expression analysis from the same patient are compared.
2. The pathway activation profiles (PAS) on a tissue specific level using methods described in (Buzdin et al., Front. Mol. Biosci. 1:8. 2014, Borisov et al., 2014).
3. It is determined which pathways are pathologic and what pathways are good for the cell.
4. For the pathways that are pathologic, the optimal combination of drugs are determined (drugs with known or predicted molecular targets or effects on transcriptomes), which minimize the difference between the different pathway activation states.
5. Minimize, where possible, toxicity and adverse effects.
6. The drug combinations, which work best and have least toxic and adverse effects in most tissues (especially in the long-lived cells) are determined as being the best for the individual patient.

When screening for gero-protector (drugs that slow or prevent the pathologic age-related changes or repair accumulated damage) the combination must work well in long-lived cells and tissues like the brain, muscle, stem cells. Some non-limiting examples of these exact algorithms for these combinations are described below.

1. Drug Scoring for their Ability to Compensate the Pathological Changes in the Signaling Pathways

The following method is used for predictive assessment of drug efficiency for individual patients based on their ability to compensate the pathological changes in the plethora of signaling pathways (signalome). For example, for the inhibitor drugs the following scheme was proposed.

$$DS {1}_{d} = {\sum }_{t} {DTI}_{dt} {\sum }_{p}^{ } {NII}_{tp} \cdot {AMCF}_{p} \cdot {PAS}_{p} ,$$

where the pathway activation strength, PAS, is

$${PAS}_{p} = {\sum }_{n} {ARR}_{np} \cdot {BTIF}_{n} \cdot 1 g ( {CNR}_{n} ) .$$

Here CNRₙ is the case-to-normal ratio, which is equal to ratio of expression levels for a gene n in a given patient and the average normal level in the population,

$${BTIF}_{n} = \{ \begin{matrix}0 , {CNR}_{n} value lies within the tolerance interval \\ 1 , {CNR}_{n} value lies beyond the tolerance interval\end{matrix}$$

ARR is a activator/repressor role discrete flag:

$${ARR}_{np} = \{ \begin{matrix}- 1 ; protein n is a signal repressor in a pathway p \\ - 0 , 5 ; protein n is more likely s signal repressor in a pathway p \\ 0 ; the role of a protein n in a pathway p is either ambivalent \\ or neutral \\ 0 , 5 ; protein n is more likely a signal activator in a pathway p \\ 1 ; protein n is a signal activator in a pathway p\end{matrix}$$

AMCF (activation-to-mitosis conversion factor) is a discrete flag

$${AMCF}_{p} \{ \begin{matrix}- 1 , pathway activation is anti \text{-} mitotic \\ 1 , pathways activation is pro \text{-} mitotic\end{matrix}$$

The action of a (protein activity inhibitor) drug was described using the discrete drug-target index:

$${DTI}_{dt} = \{ \begin{matrix}0 , drug d inhibits protein t \\ 1 , drug d does not inhibit protein t\end{matrix}$$

The discrete flag of node involvement index is

$${NII}_{tp} = \{ \begin{matrix}0 , pathaway p does not contain the protein t \\ 1 , pathaway p contains the protein t\end{matrix}$$

For the activator drugs the DS1 function should be used with the opposite (“minus”) sign before the right-hand part.

Although this approach was previously proposed for the targeted drugs in oncology: monoclonal antibodies (a.k.a. mAbs), kinase inhibitors (a.k.a. nibs) etc., it can be extended to other fields of medicine, such as, e.g., geriatrics and used for scoring of geroprotectors according to their ability to restore the juvenile state of signaling pathways in the critical (bone marrow, epithelial, osteoblast etc.) cells of a given aged person.

2. Possible Modifications of the Formula for Drug Scoring

The formula for the DS1 value contains three discrete flags, AMCF, NII, and ARR, which may be replaced with continuous analogs to reproduce the drug action more precisely.

First, we can substitute the AMCF flags with the continuous weight factors for take into account the relative importance of different pathways in the mechanism of drug action,

$$DS {3}_{d} = {\sum }_{t}^{ } {DTI}_{dt} {\sum }_{p} {NII}_{tp} \cdot {PAS}_{p} \cdot {w}_{pd} .$$

The weighting coefficients, w_{pd}, can be chosen, e.g., using the least square (or any other) fit procedure to minimize the error function,

$${S}_{d} = {\sum }_{c} {( DS {3}_{dc} - {ClinFlag}_{dc} )}^{2} ,$$

where ClinFlag_{dc} is the discrete value that is equal to 100% and zero, respectively, if the drug application has led to the observed drug d effect in the case c, which may be either an in vitro experiment or clinical observation.
Second, if each drug affects the expression level of each gene in its own way, then the drug-target index, DTI, should be substituted with a continuous value, DTA_{dt}=1 g(DTR_{dt}). Let the DTA, drug-target action, be a value that reflects the changes of gene expression levels where the drug-target ratio DTR_{dt} is the ratio of measured expression levels for the target gene t after and before the application of the drug d.

Then, the following formula for the drug score may be suggested:

$$DS {4}_{d} = {\sum }_{t} {DTA}_{dt} {\sum }_{p} {NII}_{tp} \cdot {AMCF}_{p} \cdot {PAS}_{p} = - {\sum }_{t} 1 g ( {DTR}_{dt} ) {\sum }_{p}^{ } {NII}_{tp} \cdot {AMCF}_{p} \cdot {\sum }_{n} {ARR}_{np} \cdot {BTIF}_{n} \cdot 1 g ( {CNR}_{n} )$$

Since this summation is performed twice for the logarithmic ratios, the value above is in fact the negative covariance between drug action and pathological changes in the transcriptome level that takes into account the pathway activation/suppression and their cell proliferation consequences.

Third, we can replace the discrete ARR flags with the continuous functions of relative importance of each gene/gene product for the pathway activation. This leads to the following assessment,

$$DS {5}_{d} = {\sum }_{t} {DTI}_{dt} {\sum }_{p} {NII}_{tp} \cdot {AMCF}_{p} \cdot {\sum }_{n}^{ } {w}_{np} \cdot {BTIF}_{n} \cdot 1 g ( {CNR}_{n} )$$

As far as we have mentioned in (Buzdin, 2014), two ways for determination of wₙₚ functions may be suggested. The former operates with the concept of sensitivity of the ODE system on the free parameters (Kholodenko, 2003), which is generally applied to kinetic constants (such as the dissociation constant, the Michaelis-Menten constants etc.), but may also be used for the total concentrations of certain proteins in the kinetic model of a pathway. The latter way to calculate the importance function for the genes/proteins in a pathway is related to the stiffness/sloppiness analysis (Daniels, 2008) for the effector activation upon total protein concentrations. The eigenvector components of the Hesse matrix (that is constructed for the quadratic difference function between the calculated time-courses for the activation of the pathway effector proteins and measured, using, e.g., the Western blot technique, concentrations of the activated effectors) along the stiffest direction, may be used for assessment of the wₙₚ value.

3. Assessment of Joint Action of Multiple Drugs

The combined treatment using several drugs simultaneously leads to the problem of taking into account the synergistic action of different drugs.

Previously (Buzdin, 2014) it has been the following simplified method has been proposed and validated, which assumes the multiplicative dependence of overall outcome of pathway activation upon the expression levels of each gene in signaling pathways. The additive functions like multiple drug scores (DS1-DS5) emerge after taking the logarithm from this multiplicative value.

If assume this simplified hypothesis for the description of joint drug action (say, drugs d₁ and d₂) as well, then the overall reaction of a signaling pathway that is caused by these drugs, should also multiplicative, and, consequently, the drug score should be additive:

DS(d₁+d₂)=DS(d₁)+DS(d₁).

The presence of synergistic/anti-synergistic action of the drugs may be taken into account using the following cross-talk item Δ:

DS(d₁+d₂)=DS(d₁)+DS(d₂)+Δ=DS(d₁)+DS(d₂)+C_{syn}·sign(DS(d₁)·DS(d₂))\sqrt{|DS(d₁)·DS(d₂)|},

where C_{syn} is the synergistic constant that is equal, e.q. to 3 or 2 for strong drug synergism, 1 for moderate synergism, 0 for independent drug action, −1 for moderate anti-synergism and −2 or −3 for strong anti-synergism.

4. Assessment of Drug Toxicity

The toxicity of a drug can be evaluated using the following method

$$TOX ( d ) = {\sum }_{r} {TOX}_{r} ( d ) = {\sum }_{r} \frac{{ARD}_{r}}{1 g \frac{MTC {50}_{r} ( d )}{CAC ( d )}} ,$$

when MTC50ᵣ(d) is the maximally tolerable concentrations of a drug d according to the adverse reaction r (that causes this adverse reaction over the 50% of population),CAC (d) is the clinically acting concentration of the same drug, and ARD_{d} is the expert-assessed adverse reaction danger that may be equal to 1 for relatively tolerable and reversible reactions, and, e.g., 100, for instantly fatal consequences.

Similarly to the assessment of joint drug action, the joint toxicity of two drugs according to the adverse reaction r, can be represented as follows,

TOXᵣ(d₁+d₂)=max(0;TOXᵣ(d₁)+TOXᵣ(d₂)+C_{syn}·\sqrt{TOXᵣ(d₁)·TOXᵣ(d₂)}),

when the C_{syn} factor depends of both reaction type r and two drugs d₁ and d₂.

Taking into account the toxicity for the drug scoring may result in the following function,
DS6_{d}=w_{action}·DS(1÷5)^{d}−wₜₒₓ·TOX_{d}. The weighting factors, w_{action} and wₜₒₓ, may be defined only a posteriori (e.g., after a least squares fitting during the comparison of a DS6 values and clinical overall outcome of application of a drug d).

### EXAMPLES

### Example 1

A set of commercially available drugs for treating a specific disease are chosen. For example, FDA approved drugs for treating a specific cancer, say kidney cancer. For each of the drugs, an in silico analysis is performed to evaluate several characteristics of each drug in database 180 (FIG. 1A):
a. mechanism of action (signaling and metabolic pathways significantly dysregulated by a given drug);
b. drug resistance (a list of pathways correlated with drug resistance in cell lines);
c. biomarker identification (when gene expression dataset for certain clinical trial was available we determined what pathways could serve as biomarkers); and
d. drug repurposing (a prioritized list of different types of cancer the drug might be effective against).

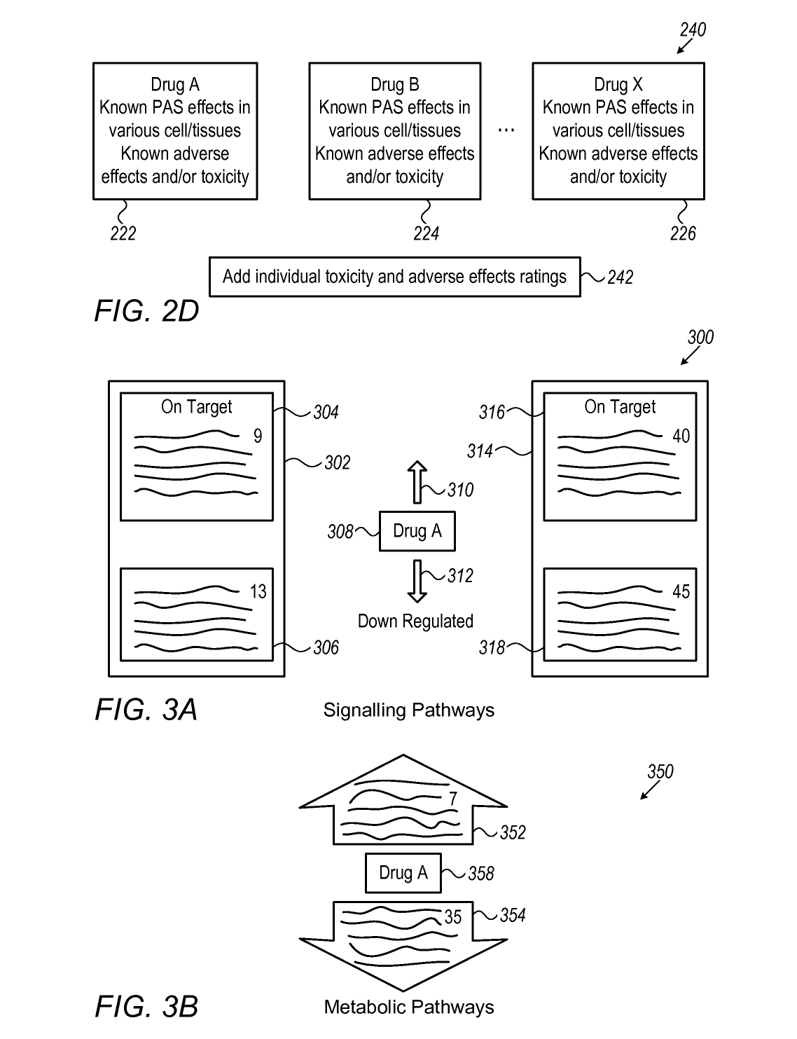

Reference is now made to FIGS. 3A-3B, which are simplified diagrams of effects of a drug on up-regulating and down-regulating signaling pathways 300 and metabolic pathways 350, respectively, in accordance with embodiments of the present invention A drug—say a tyrosine kinase inhibitor “DRUG A”, 308 is chosen.
1. The signaling pathways 300 associated the target of the drug (say tyrosine kinase)” are termed herein “ON target” 302 and those not associated with the drug are termed herein “OFF target” 314 (FIG. 3A). Drug A, 308 is found to induce up-regulation 310 and down-regulation 312 of different pathways. For example, Drug A up-reglates a set 304 of on-target pathways and a down-regulates set 306 of on-target pathways. For example, Drug A further up-reglates a set 316 of off-target pathways and a down-regulates set 318 of off-target pathways.
2. Metabolic pathways 350 are mapped (FIG. 3B) for DRUG A.
3. Drug A, 358 up-reglates a set 352 of metabolict pathways and a down-regulates set 354 of metabolic pathways.
4. Toxicity data are mapped for DRUG A using data from database 180 (FIG. 1B) and FIGS. 2B and 2C.
1) 5) Thereafter, positively and negatively correlated with drug resistance pathways are ranked for DRUG A, as described herein. Steps 1-5 are repeated for drugs B, C, D to . . . Z.
2) The drugs are ranked according to their drug score and the one with the highest score is suggested for use in treating the kidney cancer for a population of patients.
3) If drug A is the current drug used for treating kidney cancer, but it is found that drug B has a much higher drug score than drug A, then it is recommended that drug B is used to treat the kidney cancer.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

Bauer-Mehren, A., Furlong, L. I., Sanz, F. (2009). Pathway databases and tools for their exploitation: benefits, current limitations and challenges. Mol Syst Biol, 5, article 290. doi: 10.1038/msb.2009.47.

Birtwistle, M. R., Hatakeyama, M., Yumoto, N., Ogunnaike, B. A. et al. (2007). Ligand-dependent responses of the ErbB signaling network: experimental and modeling analyses. Mol Syst Biol, 3, article 144. doi: 10.1038/msb4100188.

Borisov, N., Aksamitiene, E., Kiyatkin, A., Legewie, S. et al. (2009). Systems-level interactions between insulin-EGF networks amplify mitogenic signaling. Mol Syst Biol, 5, article 256, 2009. doi: 10.1038/msb.2009.19.

Borisov, N. M., Chistopolsky, A. S., Faeder, J. R., Kholodenko, B. N. (2008). Domain-oriented reduction of rule-based network models. IET Syst Biol, 2, 342-351. doi: 10.1049/iet-syb: 20070081.

Borisov, N. M., Markevich, N. I., Hoek, J. B., Kholodenko, B. N. (2006). Trading the micro-world of combinatorial complexity for the macro-world of protein interaction domains. BioSystems, 83, 152-166. doi: 10.1016/j.biosystems.2005.03.006.

Borisov, N. M., Terekhanova, N. V., Aliper, A. M., Venkova, L. S., Smirnov, P. Y., Roumiantsev, S., . . . & Buzdin, A. A. (2014). Signaling pathway activation profiles make better markers of cancer than expression of individual genes. Oncotarget, 5.

Buzdin A A, Zhavoronkov A A, Korzinkin M B, Roumiantsev S A, Aliper A M, Venkova L S, Smirnov P Y and Borisov N M (2014) The OncoFinder algorithm for minimizing the errors introduced by the high-throughput methods of transcriptome analysis. Front. Mol. Biosci. 1:8. doi: 10.3389/fmolb.2014.00008, http://journal.frontiersin.org/Journal/10.3389/fmolb.2014.00008/full

Buzdin, A. A., Zhavoronkov, A. A., Korzinkin, M. B., Venkova, L. S., Zenin, A. A., Smirnov, Ph. Yu. and N. M. Borisov. OncoFinder, a new method for the analysis of intracellular signaling pathway activation using transcriptomic data. Frontiers in Genetics: Bioinformatics and Computational Biology, 2014, doi: 10.3389/fgene.2014.00055.

Conzelmann, H., Saez-Rodriguez, J., Sauter, T., Kholodenko, B. N., Gilles E. D., (2006). A domain-oriented approach to the reduction of combinatorial complexity in signal transduction networks. BMC Bioinformatics, 7, article 4. doi: 10.1186/1471-2105-7-34.

Daniels, B. C., Chen, Y. J., Sethna, J. P., Gutenkunst, R. N., Myers, C. R. (2008). Sloppiness, robustness and evolvability in systems biology. Curr Opin Biotechnol, 19, 389-395. arXiv: 0805.2628v1

Elkon, R.,Vesterman, R., Amit, N. (2008). SPIKE—a database, visualization and analysis tool of cellular signaling pathways. BMC Bioinformatics, 9, article 110: doi: 10.1093/nar/gkq1167.

GEO repository URL: http://www.ncbi.nlm.nih.gov/geo/

Haw, R., Stein, L. (2012). Using the Reactome database. Curr Protoc Bioinformatics, June, chapter 8, unit 8.7. doi: 10.1002/0471250953.bi0807s38.

Kholodenko, B. N., Demin, O. V., Moehren, G., Hoek, J. B. (1999). Quantification of short term signaling by the epidermal growth factor receptor. J Biol Chem, 274, 30169-30181. doi: 10.1074/jbc.274.42.30169.

Kholodenko, B., Kiyatkin, A., Bruggeman, F., Sontag, E., et al. (2003). Untangling the wires: a strategy to trace functional interactions in signaling and gene networks, Proc Natl Acad Sci, 20, 12841-12846. doi: 10.1073/pnas.192442699.

Kholodenko, B. N., Demin, O. V., Moehren, G, and. J. B. Hoek. (1999) Quantification of Short Term Signaling by the Epidermal Growth Factor Receptor. J. Biol. Chem, vol. 274, pp. 30169-81.

Kiyatkin, A., Aksamitiene, E., Markevich N. I., Borisov, N. M. et al. (2006). Scaffolding protein GAB1 sustains epidermal growth factor-induced mitogenic and survival signaling by multiple positive feedback loops. J Biol Chem, 281, 19925-19938. doi: 10.1074/jbc.M600482200.

Kuzmina, N. B., Borisov, N. M. Handling complex rule-based models of mitogenic cell signaling (On the example of ERK activation upon EGF stimulation). (2011). Intl Proc Chem Biol Envir Engng, 5, 76-82.

Mathivanan, S., Periaswamy, B., Gandhi, T., Kandasamy, K. et at. (2006). An evaluation of human protein-protein interaction data in the public domain. BMC Bioinformatics, 7, article S19. doi: 10.1186/1471-2105-7-S5-S19.

Nakaya, A., Katayama, T., Itoh, M., Hiranuka, K. et al.(2013). KEGG OC: a large-scale automatic construction of taxonomy-based ortholog clusters. Nucleic Acids Res, Jan, 41. doi: 10.1093/nar/gks1239.

Nikitin, A., Egorov, S., Daraselia, N., Mazo, I. (2003). Pathway studio—the analysis and navigation of molecular networks. Bioinformatics, 19, 2155-2157. doi: 10.1093/bioinformatics/btg290.

SABiosciences, a Qiagen company. URL: http://www.sabiosciences.com/pathwaycentral.php

The UniProt consortium. (2011). Ongoing and future developments at the Universal Protein Resource.Nucleic Acids Research, 39, D214-D219. doi: 10.1093/nar/gkq1020.

Yizhak K., Gabay O., Cohen H., Rupin E. (2013). Model-based identification of drug targets that revert disrupted metabolism and its application to ageing. Nature Communications, 4, 2632-doi: 10.1038/ncomms3632.

Zhavoronkov A, Buzdin A A, Garazha A V, Borissov N M and Moskalev A A (2014) Signaling pathway cloud regulation for in silico screening and ranking of the potential geroprotective drugs. Front. Genet. 5:49. doi: 10.3389/fgene.2014.00049

1. A method for improving drug efficacy and safety for treating a disorder in a patient, the method comprising:
a. providing a drug score database (DSD) based on pathway manifestation strengths (PMSs) for a plurality of biological pathways associated with the drug in the treatment of the disorder; and
b. comparing said pathway manifestation strengths of said plurality of biological pathways of said patient with said drug score database to provide a predictive indication if said patient is a responder or non-responder to said drug to determine whether said drug should be used in treating said patient.

2. A method according to claim 1, wherein said providing a drug score database (DSD) step comprises:
a. obtaining proliferative bodily samples and healthy bodily samples from patients;
b. applying said drug to said patients; and
c. determining responder and non-responder patients to said drug.

3. A method according to claim 2, wherein said determining step comprises comparing gene expression in selected signaling pathways.

4. A method according to claim 3, wherein said selected signaling pathways are associated with said drug.

5. A method according to claim 2, wherein said determining step further comprises determining a drug score at least one pathway manifestation strength (PMS) value for each pathway in said responder and said non-responder patients.

6. A method according to claim 5, wherein said determining step further comprises determining a drug score for said drug based on said at least one pathway manifestation strength (PMS) value.

7. A method according to claim 2, wherein said bodily samples are selected from the group consisting of a tissue sample, a cell culture, an individual single cell, a bodily sample, an organism sample and a microorganism sample.

8. A method according to claim 1, wherein said biological pathways are signaling pathways.

9. A method according to claim 3, wherein said biological pathways are metabolic pathways.

10. A method according to claim 3, wherein said gene expression comprises quantifying expression of plurality of gene products.

11. A method according to claim 10, wherein said gene products comprises a set of at least five gene products.

12. A method according to claim 11, further comprising:
d. calculating a pathway activation strength (PAS), indicative of said pathway activation of each of said biological pathways.

13. A method according to claim 12, wherein said calculating step comprises adding concentrations of said set of said at least five gene products of said sample and comparing to a same set in said at least one control sample.

14. A method according to claim 13, wherein said at least one function comprises an up-regulating function and a down-regulating function.

15. A method according to claim 1, wherein said patient suffers from a proliferative disease or disorder.

16. A method according to claim 15, wherein said proliferative disease or disorder is cancer.

17. A computer software product, said product configured for predicting drug efficacy for treating a disorder in a patient, the product comprising a computer-readable medium in which program instructions are stored, which instructions, when read by a computer, cause the computer to:
a. provide a drug score database (DSD) based on pathway manifestation strengths (PMSs) for a plurality of biological pathways associated with the drug in the treatment of the disorder; and
b. compare said pathway manifestation strengths of said plurality of biological pathways of said patient with said drug score database to provide a predictive indication if said patient is a responder or non-responder to said drug to determine whether said drug should be used in treating said patient.

18. A system for predicting drug efficacy for treating a disorder in a patient the system comprising:
a. a processor adapted to activate a computer-readable medium in which program instructions are stored, which instructions, when read by a computer, cause the processor to:
i. provide a drug score database (DSD) based on pathway manifestation strengths (PMSs) for a plurality of biological pathways associated with the drug in the treatment of the disorder; and
ii. compare said pathway manifestation strengths of said plurality of biological pathways of said patient with said drug score database to provide a predictive indication if said patient is a responder or non-responder to said drug to determine whether said drug should be used in treating said patient;
b. a memory for storing said drug score database (DSD); and
c. a display for displaying data associated with said predictive indication of said patient.

19. A method according to claim 18, wherein said drug, previously used for a first indication is used for a new second indication.

20. A method according to claim 19, wherein said drug is at least one of repurposed and repositioned.
